(19) United States
(12) Patent Application Publication
(10) Pub. No.: US 2025/0288445 A1
(43) Pub. Date: Sep. 18, 2025

(54) BABY EAR DEFORMITY CORRECTION KIT

(71) Applicant: Yasar Hüseyin ONGANLAR, Ankara (TR)

(72) Inventor: Yasar Hüseyin ONGANLAR, Ankara (TR)

(21) Appl. No.: 18/837,193

(22) PCT Filed: Aug. 10, 2022
(86) PCT No.: PCT/TR2022/050837

§ 371 Date: Mar. 24, 2025

(30) Foreign Application Priority Data
Feb. 9, 2022 (TR) 2022/001685

(51) Int. Cl. A61F 5/01; A61F 11/00

(52) U.S. Cl. CPC A61F 5/01; A61F 11/00

(57) ABSTRACT

The present invention relates to a baby ear deformity correction kit (1), which is used to correct deformity or deformity of the ears caused by genetic and/or acquired factors in the ears of new-born babies. The present invention allows intervention without the need for surgery to eliminate the relevant deformity using the main parts needed according to the deformed region or regions of the ear, such as the cradle (4)/bed, cap (2), outer ear ring retractor part (5) and conchal former (6) which are the main parts of the baby ear deformity correction kit (1).

### FIELD OF THE INVENTION

The present invention relates to a baby ear deformity correction kit, which is used to correct deformity or deformity of the ears caused by genetic and/or acquired factors in the ears of new-born babies.

The present invention allows intervention without the need for surgery to eliminate the relevant deformity using the main parts needed according to the deformed region or regions of the ear, such as the cradle/bed, cap, outer ear ring retractor part and conchal former which are main parts of the baby ear deformity correction kit.

### PRIOR ART

Today, there are ear deformation correction devices used to correct deformities in the ears of new-born babies, however, since the existing apparatus and apparatus parts are not functional enough, they are insufficient in terms of functionality in terms of local correction of the deformed areas of the ear.

In the document US2010204793, which is related to the subject in the art, an ear deformity correction apparatus available in new-born babies is mentioned. The way it works is different from our present invention and it is possible that it is insufficient in terms of functionality since the relevant apparatus does not contain a part that serves this purpose in eliminating the disorders present in the earlobe of new-born babies and due to the fact that the part cannot be removed in treatments that do not require because the fixation of the part in the middle of the apparatus, which gives the inner ring of the ear (antihelix) correction by pressing the auricle from the back, and gives the Y form (crus antihelix), which should be at the end of the inner ring, to the relevant apparatus.

A similar system with a new-born baby ear deformity removal apparatus is mentioned in document CN203693851 which is another document related to the subject in the art, but the way it works is different from our present invention. Again, new spaces are created in the remaining areas of the ear shaper kit, apart from the connection points of the bed with the cap, from there, new areas are created in order to fix the ear shaper kit to the skin from the outside of the bed when the fixation and attachments are placed in the space in the outer ring, especially in the outer ring defects of the ear, and these spaces are also formed and thus they do not appear in the previous design. In the previous design, it was practically noticed in use cases that the double-sided tape on the edges of the ear cradle does not completely fulfil this function.

In current applications, it is not mentioned that the new-born baby ear deformity devices in the abovementioned documents provide treatment for earlobe disorders and the inner ring is corrected with the help of the pressure it exerts on the auricle from below and it is not also mentioned that the part used to give the Y form at the end of the inner ring is designed in such a way that it can be inserted and removed when not needed, depending on the treatment method to be used.

### DESCRIPTION OF THE INVENTION

A baby ear deformity correction kit is realized tofulfil the aims of the present invention is shown in the figures.

In these figures;

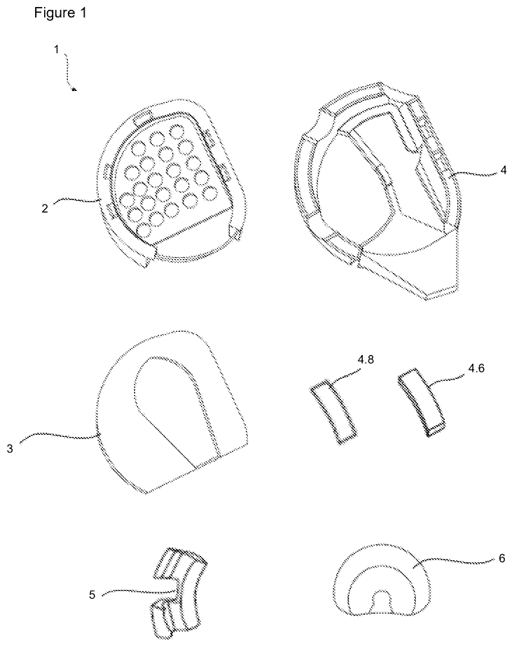

FIG. 1 is a perspective view of an exploded form of the inventive baby ear deformity correction kit.

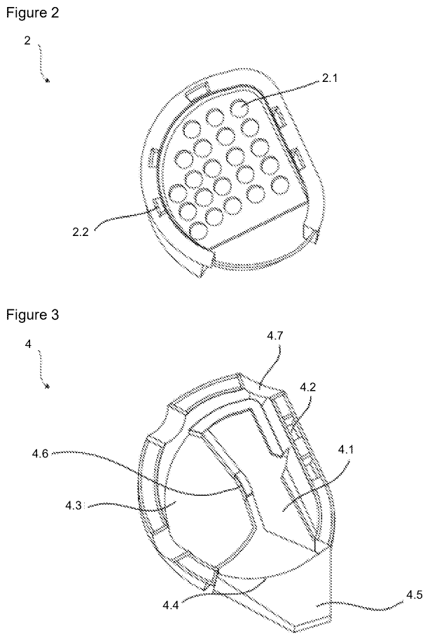

FIG. 2 is a perspective view of the cap.

FIG. 3 is a perspective view of the support part of the cradle attached to the cradle.

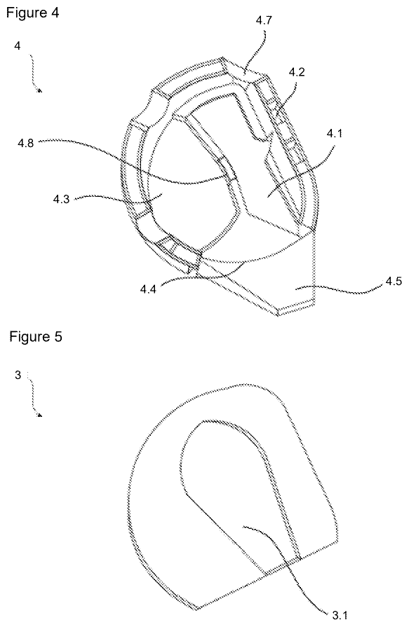

FIG. 4 is a perspective view of the spacer of the cradle attached to the cradle.

FIG. 5 is a perspective view of the double-sided tape.

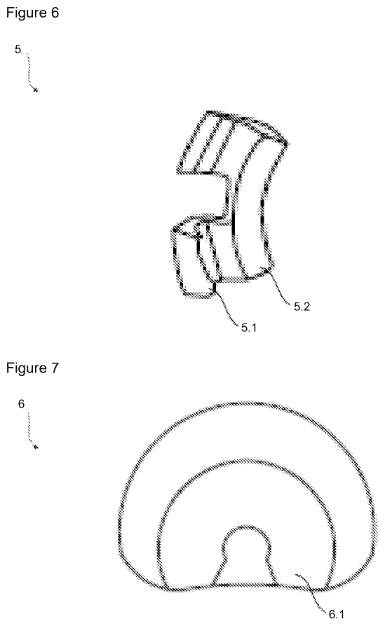

FIG. 6 is a perspective view of the retractor part.

FIG. 7 is a perspective view of the conchal former.

The parts that constitute the inventive baby ear deformity correction kit are enumerated as follows in the attached figures:
1—Baby ear deformity correction kit
2—Cap
   2.1 Air Holes
   2.2—Tab
3—Double-sided tape
   3.1—Hole
4—Cradle
   4.1—Gap
   4.2—Tab Slot
   4.3—Ear Inlet Cavity
   4.4—Protrusion
   4.5—Earlobe Shaper
   4.6—Support part
   4.7—Cavity
   4.8—Spacer piece
5—Retractor part
   5.1—Foot
   5.2—Adhesive Tape
6—Conchal Former
   6.1—Inner Filling

The inventive baby ear deformity correction kit (1) comprises of the following;
Cap (2), which has air holes (2.1) that helps the baby's ear to get fresh air and that has a plurality of tabs (2.2) for the fixation process after placing the baby's ear in the baby ear deformity correction kit (1),
Double-sided tape (3) that has a hole (3.1) allowing the same to pass through the baby's ear and reach the baby ear deformity correction kit (1), that fixes the baby ear by sticking it to the scalp behind the ear and to the baby ear deformity correction kit (1) with the help of its adhesiveness on both surfaces,
Cradle (4) that has a space (4.1) providing access to the double-sided tape (3), has a plurality of tab slots (4.2) in which the tabs (2.2) are fixed by being inserted, has an ear inlet cavity (4.3) through which the baby's ear is passed, in which the baby ear is placed,
Retractor part (5) that has two feet (5.1), one for the baby's ear and the other one used by putting it on the double-sided tape (3) through the gap (4.1), helps the treatment by putting pressure on the deformed part of the baby's ear in the lidding deformity,
Earlobe shaper (4.5) that has a protrusion (4.4) added to the cradle (4), which enables the treatment of earlobe deformities and allows shaping the earlobe in a way that does not distort its anatomy, that is attached to the earlobe,
Support part (4.6) that is being one of the parts of the cradle (4) and provides the opportunity to be used according to the condition of the treatment with the help of its removable feature, allows the Y form to be given at the end of the inner ring of the ear and the correction of the inner ring with the help of the pressure it makes on the ear from below,
A plurality of cavities (4.7) that are opened on the edges of the cradle without a tab slot (4.2) and made when necessary, especially during the correction of the lidding deformity and, if necessary, other deformity types, provides access of auxiliary materials such as plaster, which plays a role in fixing the materials used to the outside with the outward banding process, from inside the ear deformity correction kit (1) to the outside and allows the baby's ear to take fresh air,
Spacer piece (4.8) designed in the size of the support part (4.6), which is worn to prevent irritation of the baby's ear in treatments where the support part (4.6) is removed from the cradle (4) because it is not needed to be used,
Adhesive tape (5.2), which is attached to the lower surface of the two feet (5.1) of the retractor part (5), which prevents slipping that may occur due to the movements of the baby,
Internal filling (6.1) that is added to ensure that the conchal former is suitable for the ear anatomy.

The inventive baby ear deformity correction kit (1) allows the treatment of deformity of babies with congenital deformity in the earlobe without disturbing the anatomy of the earlobe with the help of the earlobe shaper (4.5) belonging to the cradle (4) part and the protrusion (4.4) on it.

Since the support part (4.6) belonging to the cradle (4) part of the inventive baby ear deformity correction kit (1) is detachable, it provides an economical and practical use as it can be attached if needed according to the condition of the deformed ear of a baby, and because it is not fixed to the cradle (4) when it is not needed, it can be used by removing it instead of cutting it out. In addition, a comfortable use is provided for the baby's ear with the help of the spacer piece (4.8) used in cases where the support part (4.6) will not be used.

It not only prevents sweating by allowing the baby's ear to breathe, but also allows some materials used when necessary in the treatment of other deformities, especially lidding, to be fixed to the relevant parts of the baby's ear with the protruding taping technique with the help of the cavities opened on the edges of the cradle (4) where there is no tab slot (4.2), which belongs to the inventive baby ear deformity correction kit (1).

It ensures that the treatment is done correctly and shortens the treatment time by preventing the shifts that may occur due to baby movements and affect the treatment negatively by adding adhesive tape (5.2) to the contacting lower surfaces of the feet (5.1), which acts as a bridge between the baby ear and the double-sided tape (3) through the cavity (4.1) of the retractor part (5) and fixes the ear.

During the treatment, the treating physician does not have to manually fill the inner middle part of the conchal former (6) with paste and similar materials, thus ensuring that the treatment is faster and more practical by making the conchal shaper (6) suitable for the ear anatomy thanks to the inner filling (6.1).

The retractor (5) is used in helical rim and other deformities as well as lidding deformity. It serves to fix the outer ring (helix) in ear deformities.

1. A baby ear deformity correction kit (1) of the present invention comprising of a cap (2), which has air holes (2.1) that helps the baby's ear to get fresh air and that has a plurality of tabs (2.2) for the fixation process after placing the baby's ear in the baby ear deformity correction kit (1), a double-sided tape (3) that has a hole (3.1) allowing the same to pass through the baby's ear and reach the baby ear deformity correction kit (1), that fixes the baby ear by sticking it to the scalp behind the ear and to the baby ear deformity correction kit (1) with the help of its adhesiveness on both surfaces, a cradle (4) that has a space (4.1) providing access to the double-sided tape (3), has a plurality of tab slots (4.2) in which the tabs (2.2) are fixed by being inserted, has an ear inlet cavity (4.3) through which the baby's ear is passed, in which the baby ear is placed, a retractor part (5) that has two feet (5.1), one for the baby's ear and the other one used by putting it on the double-sided tape (3) through the gap (4.1), helps the treatment by putting pressure on the deformed part of the baby's ear in the lidding deformity, and characterized in that the present invention comprisesan earlobe shaper (4.5) that has a protrusion (4.4) added to the cradle (4), which enables the treatment of earlobe deformities and allows shaping the earlobe in a way that does not distort its anatomy, that is attached to the earlobe, a support part (4.6) that is being one of the parts of the cradle (4) and provides the opportunity to be used according to the condition of the treatment with the help of its removable feature, allows the Y form to be given at the end of the inner ring of the ear and the correction of the inner ring with the help of the pressure it makes on the ear from below, a plurality of cavities (4.7) that are opened on the edges of the cradle without a tab slot (4.2) and made when necessary, especially during the correction of the lidding deformity and, if necessary, other deformity types, provides access of auxiliary materials such as plaster, which plays a role in fixing the materials used to the outside with the outward banding process, from inside the ear deformity correction kit (1) to the outside and allows the baby's ear to take fresh air, a spacer piece (4.8) designed in the size of the support part (4.6), which is worn to prevent irritation of the baby's ear in treatments where the support part (4.6) is removed from the cradle (4) because it is not needed to be used, an adhesive tape (5.2), which is attached to the lower surface of the two feet (5.1) of the retractor part (5), which prevents slipping that may occur due to the movements of the baby, an internal filling (6.1) that is added to ensure that the conchal former is suitable for the ear anatomy.
